(19) United States
(12) Patent Application Publication
(10) Pub. No.: US 2014/0045253 A1
(43) Pub. Date: Feb. 13, 2014

(54) MULTI-COMPARTMENT DEVICE FOR CELL CLONING AND METHOD OF PERFORMING THE SAME

(71) Applicant: Qian Zou, Newbury Park, CA (US)

(72) Inventor: Qian Zou, Newbury Park, CA (US)

(21) Appl. No.: 13/570,015

(22) Filed: Aug. 8, 2012

(51) Int. Cl. C12N 1/00; C12M 1/00

(52) U.S. Cl. 435/289.1; 435/317.1

(57) ABSTRACT

The problems associated with the traditional methods and devices in the field of cell cloning have been solved by the multi-compartment device and method in the present invention. The device combines the advantages of a traditional petri-dish and a traditional microplate. The multi-compartment device in the present invention comprises sidewalls, which are taller than openings of the multi-compartments. The cells in the suspension flow across the multi-compartments and seed inside the compartments during a plating process. The multi-compartment device in the present invention allows easier plating process, changing conditioned medium, and cell colony detachment and transfer. The multi-compartment device also minimizes the risk of cross-contamination during cloning process and during cell colony transfer. The invention also provides an exemplary method of using the multi-compartment device for cell cloning. In one aspect of the method, the multi-compartment device may be tilted before or after adding the cell suspension during plating process.

### BACKGROUND OF THE INVENTION

This invention relates generally to an improved biological cell cloning method and device having plurality of compartments, and more particularly, relates to the increased height of a sidewall surrounding the body of the device, such that the device allows an easy seeding of cells, changing of solutions in the device, and easy transferring of cell colonies from the device.

One important application of the method and the device is in the field of cell biology to derive a cell population from a single cell. Cell populations derived from a single cell are usually identical or similar and share many genetic and phenotypic features. Such cell populations are commonly referred to as “clones.” In cell biology research it is usually desirable to use clonal cell population because a mixed cell population will make it difficult to interpret data and determine which cell type is responsible for the particular response of interest. The method to derive a cell population from a single cell is referred to as “cell cloning.” Typically, a single cell is cultured in appropriate conditions to allow it to split many times and grow into a colony. Eventually, such colonies are transferred out to other cell culture vessels for scale-up processes and further processing.

There are typically two ways of cell cloning. One is to use a cell culture device with a large and undivided surface, such as a petri-dish. The cloning surface, which is the bottom surface of the petri-dish, is often physically or chemically treated to facilitate attachment and growth of single cells. Cell suspension is added to the petri-dish and the single cells will seed on the surface of the device. Each cell will form a colony thereafter and all the colonies are formed on the same surface of the device. It is easy to seed the cloning surface and maintain the cells using this method because the medium solution may be easily added to the device and removed. It may be difficult to transfer the colonies from the device. To transfer the colonies out, a cloning ring, typically a sterilized plastic, glass, porcelain, or stainless ring, pre-dipped in grease, is placed over a colony. The cells inside the ring are then trypsinized and transferred out. Trypsin is a type of serine protease that may hydrolyze proteins. The trypsin added to the device detaches the cells from the cloning surface to release the colonies. There are high risks of contamination associated with this method since all the colonies are formed on the same surface. The cloning ring is also problematic because it may be leaky and further create risks of contamination between cell colonies. Using the cloning ring to transfer cell colonies out is a tedious task. Since all the colonies are formed on the same surface, once the medium is removed from the device all the colonies are exposed to air. Unless these colonies can be transferred out quickly, some of them may dry out and die eventually. Although many variations of this method have been developed to facilitate the transferring of the colonies and minimize cross-contamination, these variations do not solve all the problems and often have their own drawbacks. For example, a small filter paper disc, soaked with trypsin may be placed on the colonies. A brief incubation is needed to ensure the detachment of the cells. Some cells will attach to the disc and be transferred out along with the disc. Although it may be easier to transfer the colonies, this method suffers drawbacks of low transferring efficiency and increased risk of contaminations. Another variation is to insert tiny glass pieces on the surface of the device and form the colonies thereupon. The colonies may be transferred by removing the glass pieces with colonies to new vessels. Untreated glass surface on these glass pieces, however, is not suitable for cells to attach and grow. In addition, the glass pieces may move in the device to cause contamination during handling and processing.

The other way of cell cloning is to use a microplate with micro-wells. Small aliquots of medium having limited amount of cells are added to the wells, typically by pipetting. Ideally, one cell will survive in each well and one colony will grow from that single cell in each well. To obtain single cell occupancy in the wells, multiple suspensions with different cell densities may be tried out to achieve the optimal percentage of wells occupied by single cells. This method is commonly referred to as “limited dilution.” It provides a low risk of cross-contamination since the wells of the microplate are isolated by their walls. Each colony may be transferred separately without using a cloning ring. Plating the cells, i.e. adding the cell suspension into the wells, however, is very difficult and tedious if carried out manually. It requires a researcher to add the cell suspension by pipetting it into each well of the microplate. Changing the medium in the microplate is also difficult and tedious for the same reason. Even when a multi-channel pipet is used, plating microplates with 96 or more wells and changing medium therefrom are still challenging tasks. Even though automated equipment with robots may be used to plate the cells and change the medium to improve efficiency, cost is a primary prohibitive factor. The operators to handle robots need special training for the equipment. Alternatively, cells may be plated into the microplates by a flow cytometry cell sorter which also requires a special equipment and training.

It is therefore desirable to have a device, which combines the design of a conventional petri-dish and the design of a conventional microplate. Such a device will have the advantage of the petri-dish so that plating the device is carried out by simply pouring a cell suspension into the device and letting the cell settle in the device to form cell colonies. Changing conditioned medium will also be carried out simply by aspirating the conditioned medium out from the device using a serological pipet instead of using pipet tips for individual wells.

It is also desirable that such a device will also maintain the characteristics of a conventional microplate such that the wells are separated by their own walls. Cross-contamination will be minimized because the cell colonies grow in individual wells, isolated from each other. Colonies may be released and transferred separately without drying and damaging nearby colonies.

It is also desirable to seed the wells with a gradient such that the cell suspension will have a gradient in cell density within the device, which is useful when an optimal seeding density is unknown.

### SUMMARY OF THE INVENTION

The problems associated with the conventional methods and devices in the field of cell cloning have been solved by the multi-compartment device and method in the present invention. The device combines the advantages of a conventional petri-dish and a conventional microplate. In essence, the invention comprises a method utilizing a multi-compartment device, in which biological cells will be seeded and cloned to form cell colonies in the compartments. The multi-compartment design ensures single cell seeding in individual compartments such that a colony in each compartment is formed from one single cell, in order to prevent cross-contamination of the colonies. The multi-compartment design also ensures that detachment and transferring of the cell colonies may be carried out from each compartment separately, while other colonies in adjacent compartments are still submerged under a medium solution, such that those colonies do not expose to air and dry out while the first colony is being transferred. The cell colonies may be detached together, with one addition of cell releasing reagents such as trypsin. As long as the level of the trypsin solution is below the openings of the compartments, there is negligible risk of cross-contamination by one colony flowing to the adjacent compartments and mixing with the other colonies. The device also comprises sidewalls, which are at least 0.1 millimeter taller than openings of the multi-compartments, preferably 10-20 millimeters taller than the openings of the multi-compartments. Plating the compartments, i.e., letting cells to flow in the multi-compartment device and seed inside the compartments, is very easy, in which a cell suspension may be poured directly into the device, instead of being pipetted into separate compartments. The cells in the suspension will then flow across the multi-compartments and seed inside the compartments. This is important when there is no automated equipment available for the plating process to avoid the tedious pipetting task. Changing conditioned medium is also easy, in which the conditioned medium, i.e., exhausted after being consumed by the living cells, may be aspirated out, instead of being pipetted out from individual compartments.

The multi-compartment device may have various numbers of compartments, such as 6, 12, 24, 48, 96, 192, 384, or 1536 compartments for different cell cloning purposes. The shape of the compartment may be round, rectangular, or other suitable shapes. The volume of each compartment is in a range from a few microliters to a few milliliters. The multi-compartment device may be made by quartz, glass, sapphire, Pyrex, plastics such as polymethylmethacrylate (PMMA), polycarbonate (PC), polystyrene (PS), polypropylene (PP), polyethylene (PE), or other rigid materials. If made by plastics, the multi-compartment device may be made by injection molding, blow molding, or other suitable methods.

In one embodiment of the present invention, the sidewalls of the multi-compartment device are co-manufactured with the body of the device. It may be made from the same molding process that forms the body of the multi-compartment device. The sidewalls are taller than the openings of the multi-compartments by a height ranging from a few tenth millimeter to hundreds of millimeters. The multi-compartments in the device are of the same or different sizes and spacing. The bottom surface of the multi-compartments may be treated physically or chemically to provide a surface for an easy attachment of the cells and to prevent movements of the cells during handling of the device.

In another embodiment of the present invention, the assembly of the multi-compartment device includes two separate parts, a base member with sidewalls and a body member with multi-compartments. In preparation for cell cloning, the base member and the body member will be assembled together to form the multi-compartment device. The sidewalls on the base member enclose the body member and extend upwardly to a height above the openings of the multi-compartments in the body member. The bottom surface of the multi-compartments may be treated physically or chemically to provide a surface for an easy attachment of the cells and to prevent the movements of the cells during handling of the device. There may be locking means on the sidewalls of the base member such that when the body member and the base member are assembled together, the body member is prevented from movements to minimize risks of spilling of the medium solution during handling of the device. The base member may be large enough to accommodate several multi-compartment body members. The multi-compartments in this multi-body member configuration may be of different sizes or shapes such that when the cell density in the cell suspension is unknown, there is a better chance to separate cells into individual compartments.

In yet another embodiment of the present invention, the assembly of the multi-compartment device includes two separate parts, a base member with sidewalls and a body member with multi-compartments. The compartments are in fact through-holes that have openings on both sides of the body member. In preparation for cell cloning, the base member and the body member will be assembled together to form the multi-compartment device. The sidewalls on the base member enclose the body member and extend upwardly to a height above top openings of the multi-compartments in the body member. The inner surface of the base member may be treated physically or chemically to provide a surface for an easy attachment of the cells and to prevent the movements of the cells during handling of the device. There may be locking means on the sidewalls of the base member such that when the body member and the base member are assembled together, the body member is prevented from movements vertically or horizontally to minimize risks of spilling of the medium solutions during handling of the device. There may also be seals at bottom openings of the compartments such that the cells or cell colonies do not flow from one compartment into adjacent compartments during seeding of the compartments or detachment of the cell colonies. The base member may be large enough to accommodate several multi-compartment body members. The multi-compartments in this multi-body member configuration may be of different sizes or shapes such that when the cell density in the cell suspension is unknown, there is a better chance to separate cells into individual compartments.

The present invention also provides an exemplary method of using the multi-compartment device for cell cloning. In one aspect of the method, the multi-compartment device may be tilted before or after adding the cell suspension during plating process. This is important when the cell density in the cell suspension is unknown, or when a gradient of cell density in different compartments is desired.

### BRIEF DESCRIPTION OF THE DRAWINGS

FIG. 1 illustrates a first embodiment of the multi-compartment device in the present invention, in which sidewalls of the device is co-manufactured with the rest structures of the device and extends upwardly, above the openings of the multi-compartments in the device.

FIG. 1A illustrates a cross-sectional view of the multi-compartment device in the first embodiment, showing the sidewalls of the device to above the openings of the multi-compartments.

FIG. 2 illustrates a second embodiment of the multi-compartment device in the present invention, which comprises a base member and a body member having multi-compartments, including both a separated configuration and an assembled configuration of the device.

FIG. 2A illustrates a cross-sectional view of both of the base member and the body member in the separated configuration of the multi-compartment device. The base member encloses the body member when they are assembled. The sidewalls of the base member are extended above the openings of the multi-compartments in the assembled configuration.

FIG. 3 illustrates a third embodiment of the multi-compartment device in the present invention, which comprises a base member and a body member having multi-compartments, in a separated configuration. The multi-compartments have openings at both sides of the body member to form through-holes in the body member.

FIG. 3A illustrates a cross-sectional view of both of the base member and the body member in an assembled configuration. The base member encloses the body member when they are assembled. The sidewalls of the base member are extended above the top openings of the multi-compartments in the assembled configuration.

FIG. 4 illustrates a wedge configuration of a locking means for locking the body member in the base member. The wedges press against a top surface of the body member having multi-compartments to prevent the body member from movements when the base member and the body member are assembled.

FIG. 5 illustrates a recess configuration of the locking means in the base member. The protrusions in the body member having multi-compartments engage in the recesses on the base member to prevent the body member from movements when the base member and the body member are assembled.

FIG. 5A illustrates a protrusion configuration of the locking means on the body member. The protrusions in the body member having multi-compartments engage in the recesses on the base member to prevent the body member from movements when the base member and the body member are assembled.

FIG. 6 is a flowchart to illustrate an exemplary process of cell cloning using the multi-compartment device in the present invention.

FIG. 7 illustrates a position of the multi-compartment device during plating process. The cell suspension flows above the openings of the multi-compartments, while cells are being seeded on the bottom of the compartments.

FIG. 8 illustrates a position of the multi-compartment device during a detachment and cell colony transferring process.

FIG. 9 illustrates a tilting position of the multi-compartment device during a plating process.

### DETAILED DESCRIPTION OF THE INVENTION

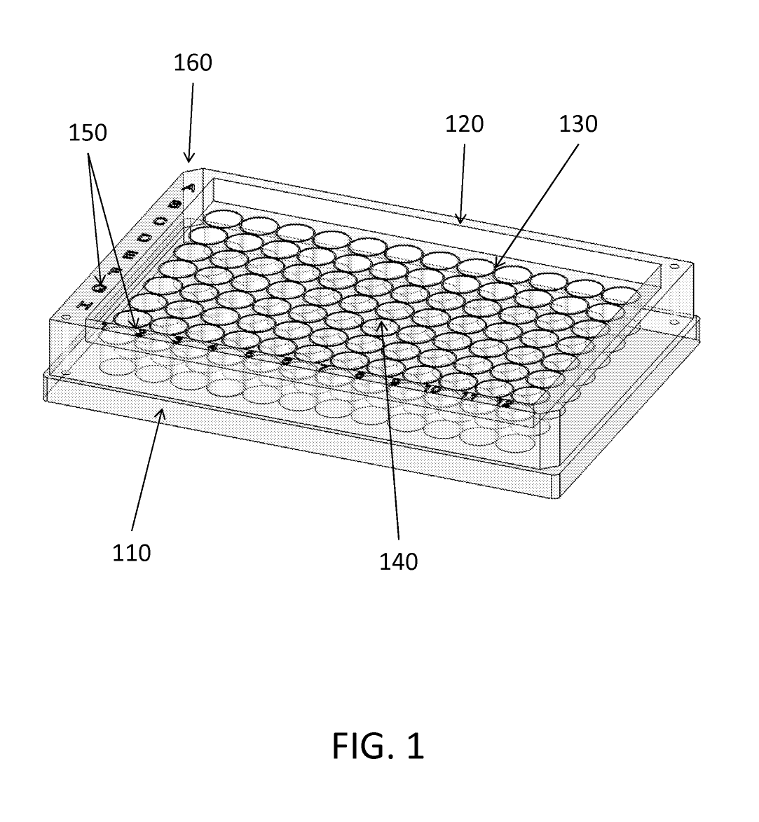

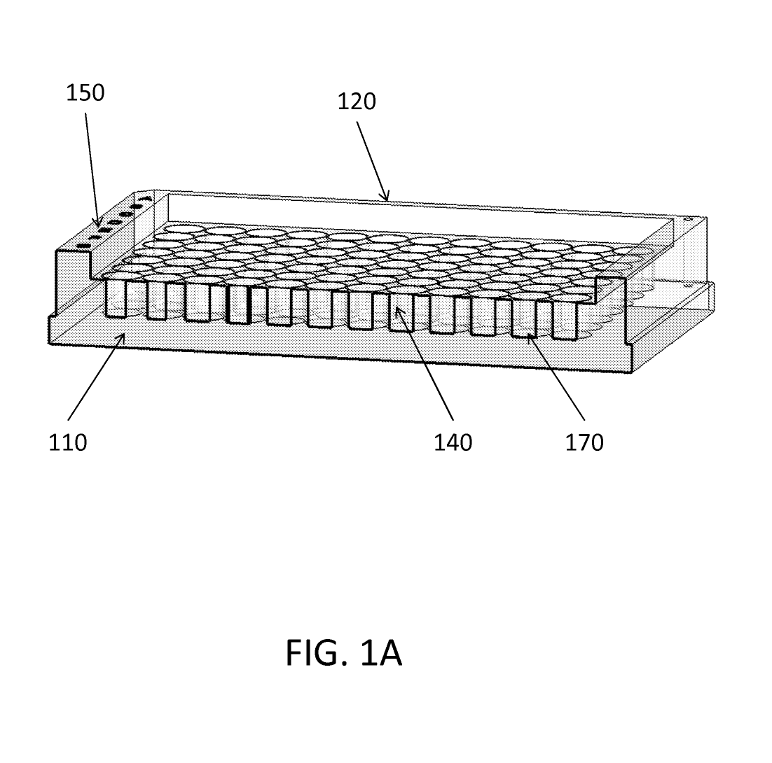

Referring to FIGS. 1 and 1A now. FIG. 1 illustrates first embodiment of the multi-compartment device in the present invention. FIG. 1A is a cross-sectional view of the multi-compartment device in the first embodiment. A multi-compartment device in this embodiment comprises a body member 110 and a sidewall 120. A bottom of the body member 110 may be slightly larger than the perimeter of the sidewall 120 for stability concerns. A top surface 130 is formed within the sidewall 120 in a position such that the top edge of the sidewall 120 is at least 0.1 millimeter taller than the top surface 130. Multi-compartments 140 are formed on the top surface 130 in the body member 110. A bottom surface of the compartment 140 is formed inside the body member 110. A top opening of the compartment 140 is formed on the top surface 130. The purpose of the taller sidewall 120 is to ensure that during a cell cloning, in a plating process for example, a cell suspension or a medium solution may overflow the compartments 140 without spilling. Plating is a process where the cell suspension is added to the multi-compartment device, after which the device is set still so that cells in the suspension settle inside the multi-compartments by gravity and attach on the cell cloning surface thereof.

The compartments 140 shown in FIG. 1 are round-shaped. In practice, such compartments may be square, oval, or rectangular-shaped or any other shape suitable for cell cloning. The depth of the compartments 140 may vary such that the volume of the compartments 140 varies from a few micro-liters to a few milliliters. FIG. 1 shows a multi-compartment device design of 96 compartments, which is typical in the industries. In practice the number of compartments may range from 6, 12, 24, 48, 96, to 1536 or even more. In a typical multi-compartment device design, a row has 2× number of compartments and a column has 3× number of compartments. Each row is typically labeled with letters 150 and each column is typically labeled with numerals 150 for identification purposes, as shown in FIG. 1. The bottom surface 170 of the compartments 140, as shown in FIG. 1A, may be treated chemically or physically to facilitate cell attachment on the surface. For example, the surface 170 may be coated with a protein D-Lysine. The cells attach to D-Lysine during growth so that they do not float when the medium solution is changed. During detachment and release of the cell colonies, a protease such as trypsin is added to the compartments. Trypsin cleaves the colonies from the bottom surface 170 of the compartments 140 by hydrolyzing the D-Lysine protein. This process is generally referred to as trypsinization.

The multi-compartment device has one or more notches 160 for orientation purpose in an automated machine, as indicated in FIG. 1. The multi-compartment device may be made of any rigid material, such as quartz, sapphire, glass, or thermoplastics. It may be opaque or transparent. If made of thermoplastics, the usual way of manufacturing is by injection molding or blow molding of polypropylene (PP), polyethylene (PE), polystyrene (PS), polycarbonate (PC), polymethylmethacrylate (PMMA), among others.

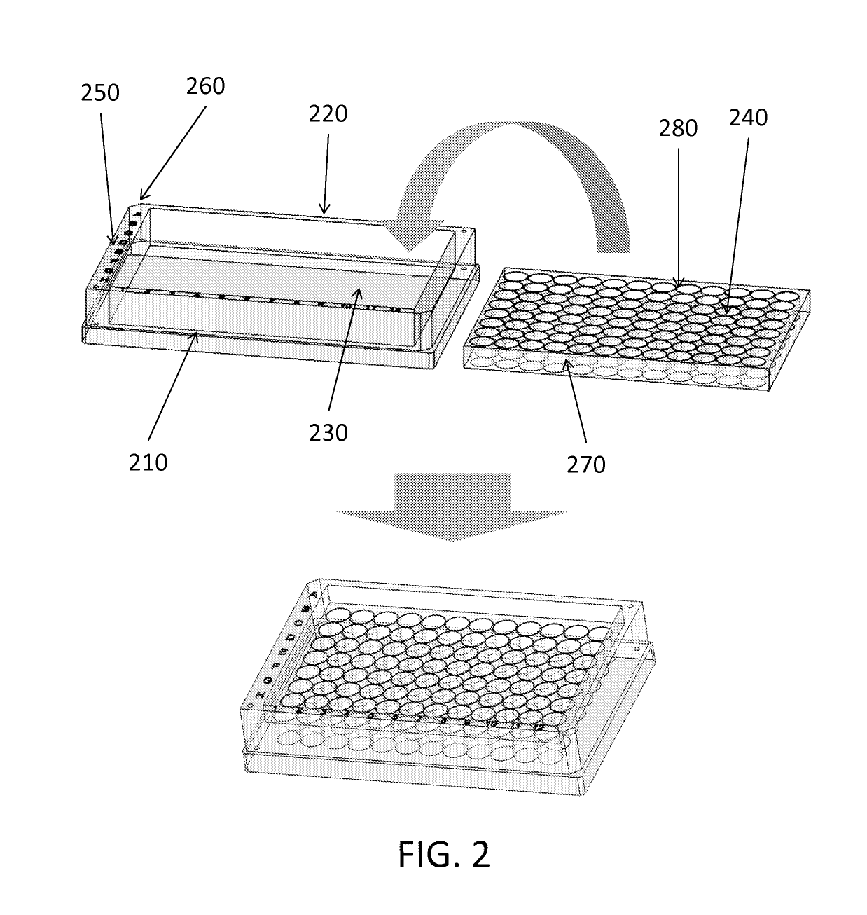

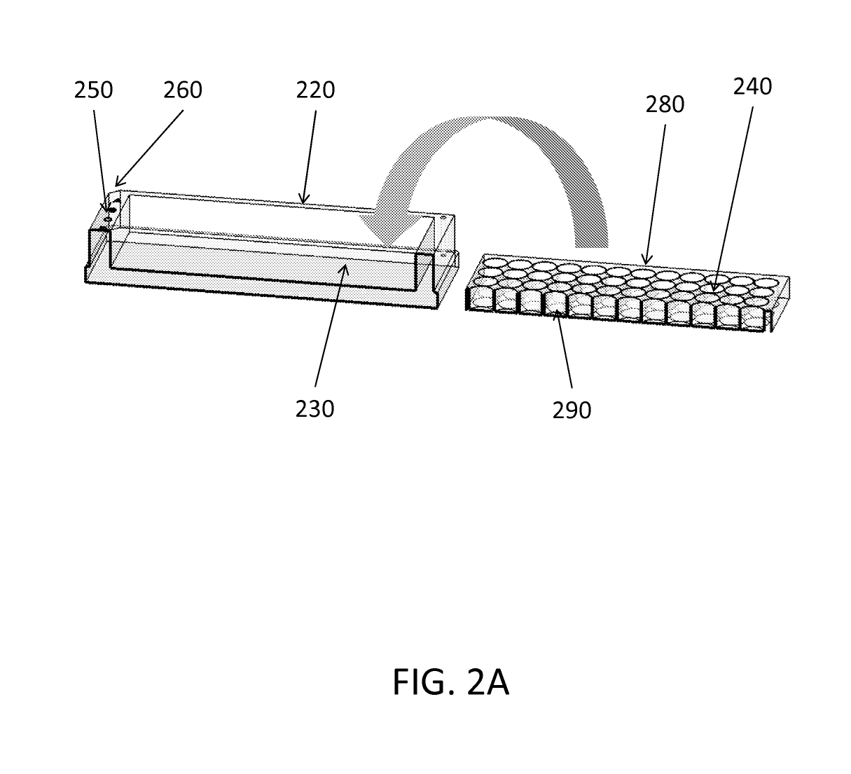

Referring to FIGS. 2 and 2A now. FIG. 2 illustrates a second or preferred embodiment of the multi-compartment device in the present invention. FIG. 2A shows a cross-sectional view of the device in the present invention. A multi-compartment device in this embodiment comprises a base member 210 and a body member 270. The base member 210 has a sidewall 220 to enclose an inner surface 230. A bottom of the base member 210 may be slightly larger than the perimeter of the sidewall 220 for stability concerns. Multi-compartments 240 are formed in the body member 270, with top openings of the compartments 240 being formed on a top surface 280 of the body member 270. A bottom surface 290 of each of the compartments 240 is formed in the body member 270 such that the compartments 240 are closed at their bottom surface 290. The base member 210 also has one or more notches 260 for orientation purpose in an automated process. The base member 210 and body member 270 may be made of the same or different materials, such as quartz, sapphire, glass, or thermoplastics like PP, PE, PS, PC, PMMA, among others. When made of the same material, they may usually be manufactured together in the same process. The number of compartments 240 in the body member 270 may be from 6, 12, 24, 48, 96, to 1536, or even more. The compartments 240 are arranged in a way such that each row has 2× number of compartments and each column has 3× number of compartments. The base member 210 has letter or numerical labels 250 on the sidewalls 220 to identify the rows and columns of the compartments 240 in the body member 270, respectively.

In preparation for cell cloning, the base member 210 and the body member 270 are assembled together to form the multi-compartment device in the present invention. The body member 270 engages in the base member 210 and is enclosed by the sidewalls 220 of the base member 210. Bottom surface of the body member 270 rests against the inner surface 230 of the base member 210. In the assembled configuration, the sidewalls 220 of the base member 210 are at least 0.1 millimeter taller than the top surface 280 of the body member 270, i.e., the top openings of the compartments 240. The sidewalls 220 may hold a cell suspension to a level above the top openings of the compartments 240 to overflow the compartments 240 during the plating process.

The compartments 240 in the body member 270 may be round-shaped, as shown in FIG. 2. The compartments 240 may also be of other shapes such as oval, rectangular, or square, among others. The bottom surface 290 of the compartments 240 in the body member 270 may be treated chemically or physically to facilitate cell attachments for cell cloning purpose. For example, the bottom surface 290 may be coated with a protein D-Lysine. The cells attach to D-Lysine during growth so that they do not float when the medium solution is changed. During detachment and release of the cell colonies, a protease such as trypsin is added to the compartments. Trypsin cleaves the colonies from the bottom surface 290 of the compartments 240 by hydrolyzing the D-Lysine protein.

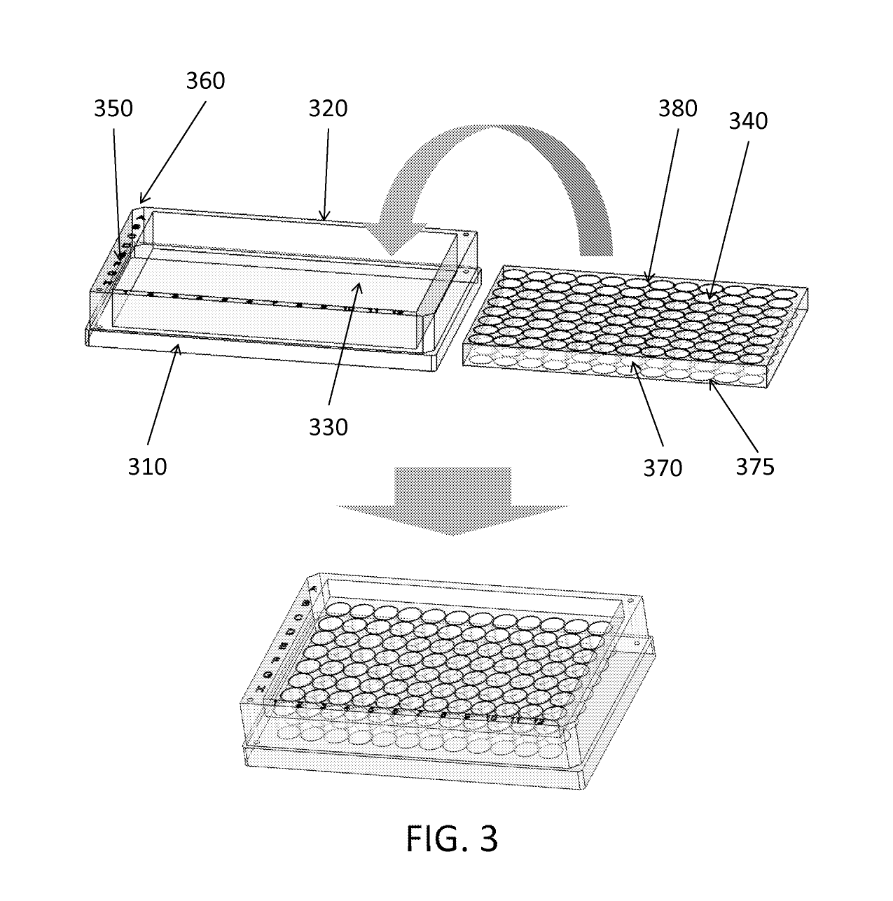

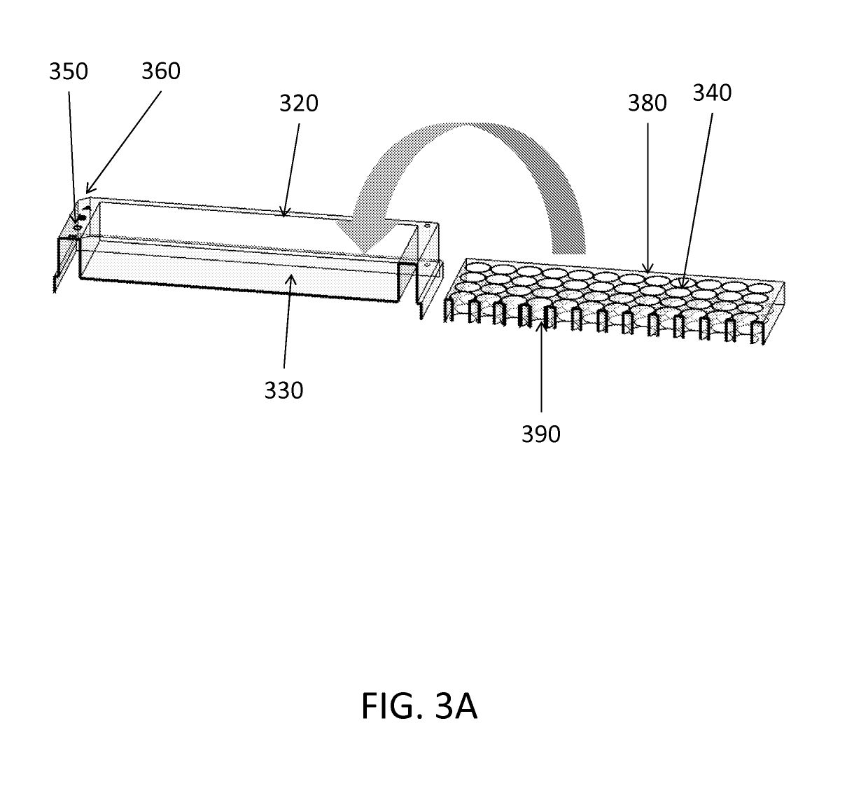

Referring to FIGS. 3 and 3A now. FIG. 3 illustrates another embodiment of the multi-compartment device in the present invention. FIG. 3A is a cross-sectional view of the multi-compartment device. Similar to the second embodiment, illustrated in FIG. 2, this embodiment also comprises a base member 310 and a body member 370. The base member 310 has a sidewall 320 enclosing an inner surface 330. The bottom of the base member 310 may be slightly larger for stability concerns. Multi-compartments 340 are formed in the body member 370 with their top openings formed on a top surface 380 of the body member 370. Instead of forming a bottom in the body member 370, the compartments 340 are open on the bottom surface 375 opposite to the top surface 380 of the body member 370 such that the compartments 340 are essentially through holes in the body member 370. The base member 310 also has one or more notches 360 for orientation purpose using an automated instrument. The base member 310 and body member 370 may be made of the same or different materials, such as quartz, sapphire, glass, or thermoplastics like PP, PE, PS, PC, PMMA, among others. When being made of the same material, they may usually be manufactured together in the same process. The number of compartments 340 in the body member 370 may be from 6, 12, 24, 48, 96, to 1536, or even more. The compartments 340 are arranged in a way such that each row has 2× number of compartments and each column has 3× number of compartments. The base member 310 has letter or numerical labels 350 on the sidewalls 320 to identify the rows and columns of the compartments 340 in the body member 370, respectively.

In preparation for cell cloning, the base member 310 and the body member 370 are assembled to form the multi-compartment device. The body member 370 engages in the base member 310 and is enclosed by the sidewalls 320 of the base member 310. The bottom surface 375 of the body member 370 rests against the inner surface 330 of the base member 310. In the assembled configuration, the sidewalls 320 of the base member 310 are at least 0.1 millimeter taller than the top surface 380 of the body member 370, i.e., the top openings of the compartments 340. The sidewalls 320 may hold a cell suspension or a medium solution to a level above the top openings of the compartments 340 to overflow the compartments 340 during a cell cloning process. During the plating process, instead of seeding the cells on the bottom surface of the compartments 340, as shown in FIG. 3, the cells seed on the inner surface 330 of the base member 310. The inner surface 330 of the base member 310 may be treated physically or chemically to facilitate cell attachments during the cell cloning process. For example, the inner surface 330 of the base member 310 may be coated with a protein D-Lysine. The cells attach to D-Lysine during growth so that they do not float when the medium solution is changed. During detachment and release of the cell colonies, a protease such as trypsin is added to the compartments. Trypsin cleaves the colonies from the inner surface 330 of the base member 310 by hydrolyzing the D-Lysine protein.

To prevent cell or cell colonies from flowing from one compartment to another, seal means may be attached to the bottom surface of the body member 370 such that when the bottom surface 375 of the body member 370 rests against the inner surface 330 of the base member 310 in the assembled configuration, the bottom openings 390 of the compartments 340 are sealed from each other. This is of importance during the plating process and the cell colony detachment process, when the cells or cell colonies may move around in the compartment and can flow into adjacent compartment to cause contamination without the seal means. It is of less importance during cloning processes when the cells are attached to the inner surface 330 of the base member 310 and immobilized.

Plating the multi-compartment device, i.e., filling the compartments with the cell suspension and letting the cells to flow to the bottom of the compartments by gravity and to attach on the cloning surface, generally involves in pouring the cell suspension directly into the device and overflowing the compartments. Because of the extended sidewalls in the present invention, the cell suspension does not spill outside. Ideally, one cell will occupy each compartment such that a single colony is formed in each compartment to prevent cross-contaminations. The multi-compartment design of the device ensures such cross-contamination may be minimized when a suspension of correct cell density is used. A colony growing from a single cell has the same genetic characteristics of the single cell, which is important in cell cloning process.

During the cloning process, the cells are typically submerged in a medium solution, which provides necessary nutrients for the cells to ensure the viability and growth of the cells. Once the medium solution is depleted, i.e., conditioned, it must be replaced with a fresh medium solution. Changing the medium solution in the multi-compartment device involves a simple aspiration of the conditioned medium solution and filling fresh medium solution to the device to a desired level to ensure the compartments overflown with the fresh medium solution. Once the conditioned medium solution in the device is aspirated, it is optional to pipet out any residual conditioned medium solution in each individual compartment before the fresh medium solution is added into the device.

After the colony is grown to a desired size, the medium solution in the multi-compartment device is discarded and a protease such as trypsin is added to detach and release the colony. Adding the trypsin also involves a simple pouring the solution directly into the multi-compartment device. Trypsin cleaves the colony from the surface where the cells have grown, so that they may be transferred out to other vessels for further processing. The multi-compartment design ensures that when the cell colonies are cleaved and floating in the protease solution, they are still confined in their individual compartments to prevent cross-contamination with colonies in adjacent compartments. Additionally, the multi-compartment design also ensures that when one colony is being transferred, the other colonies in the device are still submerged in the protease solution such that they do not expose to ambient air and dry out.

The assembly designs in the second and the third embodiments also enable a multiple body member operation for cell cloning. The base member may be made large enough to hold one or more body members. These body members may have different numbers of compartments. The compartments in these body members may have different shapes or sizes. This is important when the optimal cell density in the cell suspension is unknown such that there is a better chance to seed a single cell into a single compartment due to the large variety of the compartments of different shapes and sizes.

In the second and the third embodiments of the present invention, the multi-compartment device is formed by assembling the body member and the base member together. In the second embodiment, the bottom surface 290 of the compartments 240 is the surface to grow cell colonies. In the third embodiment, the inner surface 330 of the base member 310 is the surface to grow cell colonies. In both embodiments, the body member and the base member are separate parts and assembled together to form the multi-compartment device during the preparation of the cloning process. During the cell cloning process, solutions are added periodically into the device. The cell suspension is added at plating step. Conditioned medium solution is discarded and fresh medium solution is added periodically during the cell colony growth process. Trypsin is added during the detachment and release process. The multi-compartment device is also subject to an incubation process. Even a slight movement of the body member inside the base member may increase the risk of cross-contamination. Particularly in the third embodiment, when the inner surface 330 of the base member 310 is used for cell cloning, even a slight lateral or vertical movement of the body member 370 would result in cross-contaminations between the compartments 340 during plating or detachment processes because unattached cells or cell colonies may flow into adjacent compartments. To prevent such situation, a locking means is added in the present invention in the second and the third embodiments to prevent the movement of the body member when it is assembled to the base member.

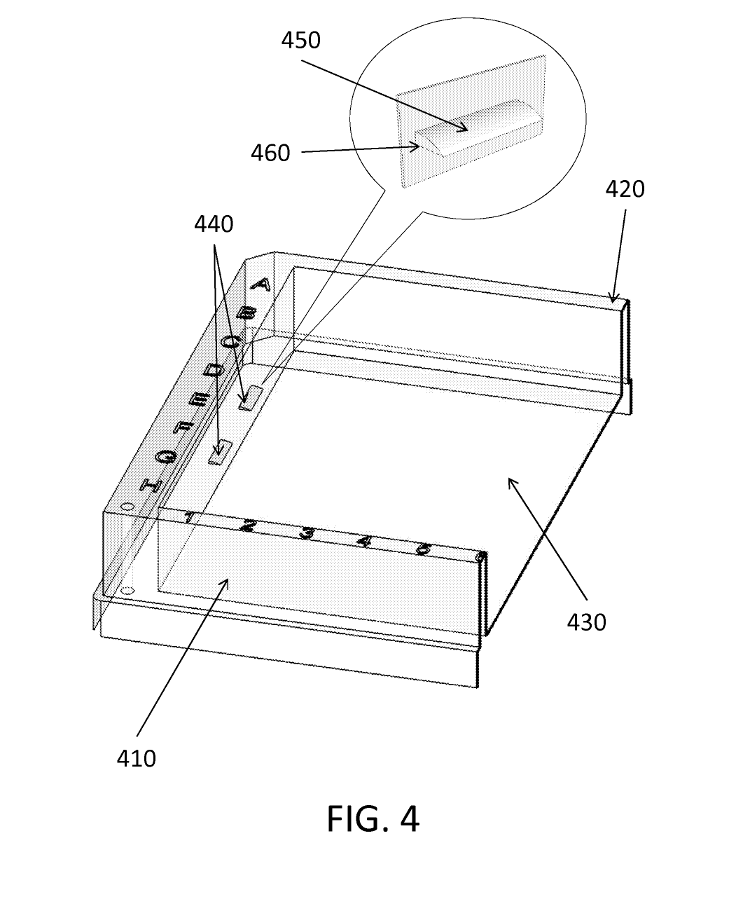

Referring to FIG. 4 now. FIG. 4 illustrates a wedge design of the locking means. The wedge 440 shown may be formed on the sidewall 420 of the base member 410, in the same process when the base member 410 is manufactured. The wedge 440 has a first surface 450 which may be curved. A second surface 460 of the wedge 440 is flat and parallel to the inner surface 430 of the base member 410, such that when the body member and the base member 410 are assembled together, the second surface 460 of the wedge 440 presses against the top surface of the body member to hold it in place and prevent its movements. The size of the wedge 440 is small enough to allow the body member to be assembled to the base member 410. During the multi-compartment device assembly, the body member is pressed against the first surface 450 of the wedge 440 into the base member 410. The pressure pushes the sidewall 420 of the base member 410 out slightly to allow the body member to move in. When the body member is fully in place, the sidewall 420 relaxes back to its original position such that the second surface 460 of the wedge 440 rests against the top surface of the body member. There may be a plurality of wedges on the perimeter of the sidewall 420 of the base member 410. The length of the wedge 440 may be as long as the length of the sidewall 420.

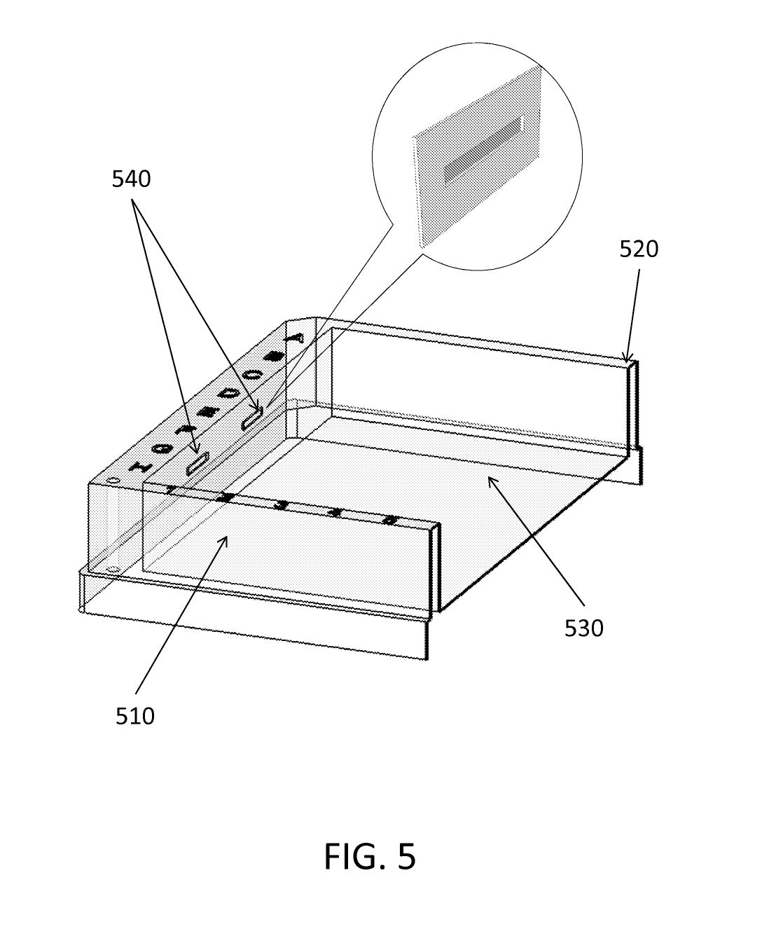

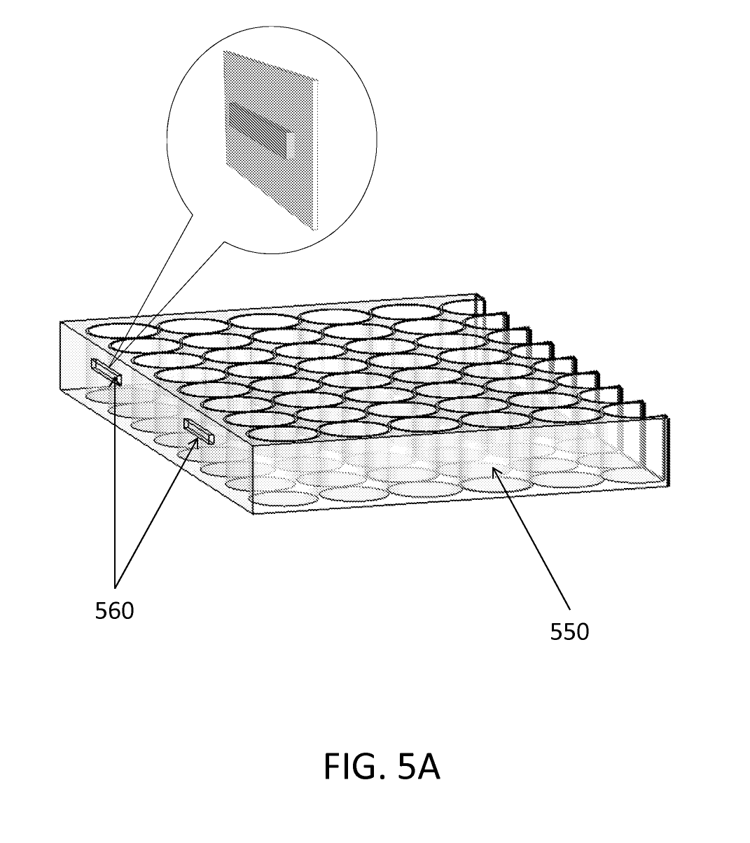

Referring to FIGS. 5 and 5A now. FIG. 5 illustrates another variation of the locking means. FIG. 5A shows the corresponding protrusions on the side of the body member. A small recess 540 is formed in the sidewall 520 of the base member 510. A small protrusion 560 is formed on the body member 550. The protrusion 560 is of the same size and shape of the recess 540. The shape of the recess 540 and the protrusion 560 may be a part of a cuboid or a cylinder. During the multi-compartment device assembly, the protrusion 560 on the body member 550 presses against the sidewall 520 of the base member 510 to push the sidewall 520 out slightly to allow the frame 550 to move in. The protrusion 560 moves into and occupies the recess 540 on the sidewall 520 of the base member 510, after which the sidewall 520 of the base member 510 to relaxes back to its original position such that the body member 550 is locked into the base member 510 against the inner surface 530 of the base member 510. Alternatively, the recess 540 may be formed on the body member 550 and the protrusion 560 may be formed on the sidewall 520 of the base member 510 to achieve the same result. The size of the recess 540 may be as long as the length of the sidewall 520. There may be a plurality of recesses on the perimeter of the sidewall 520 of the base member 510. If the size, shape, position, or the number of the recess 520 changes, the protrusion 550 on the body member must change accordingly to ensure a correct assembly of the multi-compartment device.

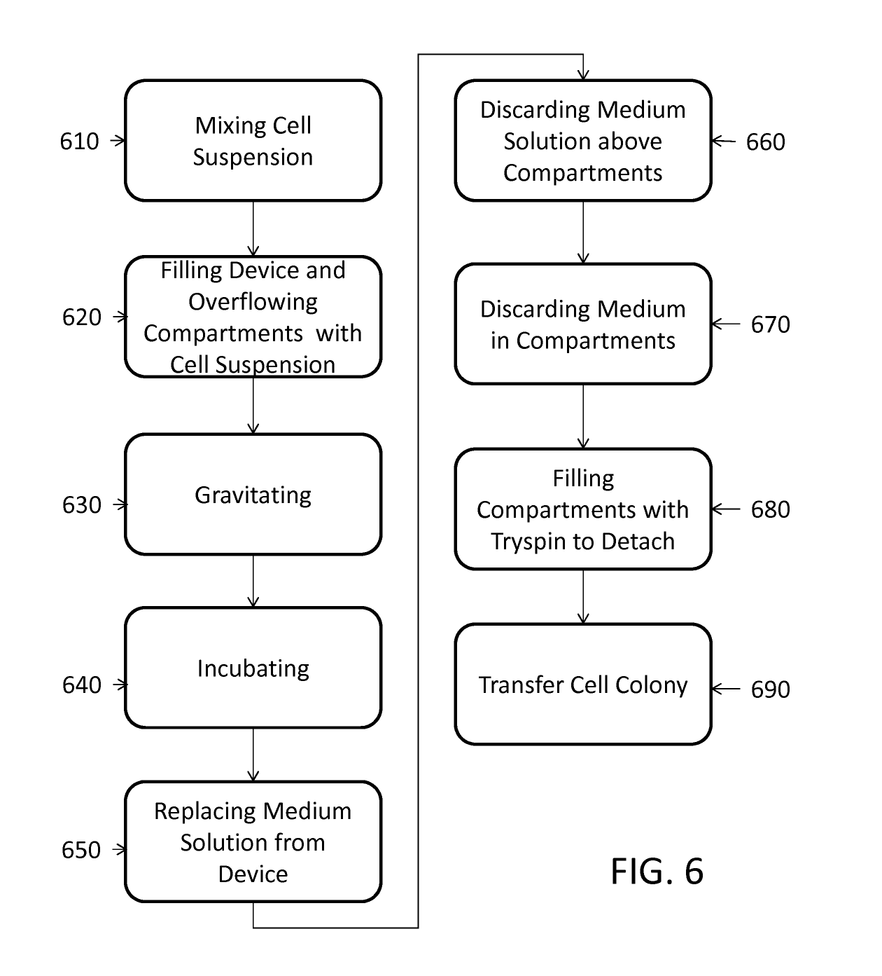

Referring to FIG. 6 now. FIG. 6 is a flowchart to illustrate a typical cell cloning process using the multi-compartment device in the present invention. A cell suspension is made by mixing living cells to a medium solution to a desired cell density in step 610.

The cell suspension is poured into a pre-assembled multi-compartment device, overflowing multi-compartments in the device to a level above top openings of the compartments in step 620. In a conventional microplate design, the cell suspension must be pipetted into each individual wells. The multi-compartment device in the present invention has an elevated sidewall, which enclosed the compartments and which allows the pouring of the cell suspension solution, thus eliminating the tedious task of pipetting. General precautions must be taken to avoid air bubbles in the compartments and to avoid foaming in the multi-compartment device.

The device is then set still for a while to let the cells to settle in the compartments by gravity (“gravitating”), and attach to the surface of the bottom surface of the compartments in step 630. In the first and the second embodiment of the present invention, the cells attach to the bottom surface of the compartments. In the third embodiment of the present invention, as discussed heretofore, the cells settle and attach onto an inner surface of the base member instead of a bottom surface of the compartments. The multi-compartment device in the present invention ensures single cell occupancy in the individual compartments such that only one colony will grow in each compartment. In a conventional petri-dish device, cells can accumulate such that several cells may grow into a single colony, which is undesirable in cell biology research.

The multi-compartment device with cells attached undergoes an incubating process in step 640, in which the device is heated up to a certain temperature in order for the cells to grow and split to form cell colonies.

Nutrients in the medium solution are consumed by the cells and must be replaced periodically in the next step 650. To replace the medium solution in the multi-compartment device, the conditioned medium solution is aspirated out and discarded. It is optional to pipet out residual medium solution in each compartment after aspiration. Fresh medium solution is poured directly into the multi-compartment device and overflown the compartments to a level above the top openings of the compartments, after which the device is returned back to incubation. It may be necessary to replace the medium solution several times until the cells split and grow into cell colonies of desired size. In a conventional microplate device, the conditioned medium solution must be pipetted out using pipet tips from individual wells instead of aspiration.

During the cell cloning process, a selection reagent may be added to the multi-compartment device. Cells non-resistant to the selection reagent are annihilated. Cells resistant to the selection reagent survive and further grow into cells colonies.

Once the cell colonies are grown to the desired size, they must be transferred out to other vessels for further processing. The multi-compartment device is removed from incubation. The medium solution is aspirated out and discarded one last time in step 660.

It is again optional to pipet out the residual medium solution from each compartment separately after aspiration, such as in step 670.

A protease such as trypsin is poured into the multi-compartment device to flow into the multi-compartments, i.e., trypsinization in step 680. Trypsin cleaves the carbonyl group of D-Lysine protein attached to the bottom surface of the compartments to detach and release the cell colonies from the bottom surface of compartments. At this stage pre-cautions must be taken such that after the compartments are filled with trypsin, the solution is at a level below the top openings of the compartments to avoid the detached cell colonies to float into adjacent compartments to contaminate the cell colonies therein. This would be difficult in a conventional petri-dish design without compartments because once the colonies are detached and released, they can float freely into each other in the trypsin solution to cause contamination.

The cell colonies are transferred one at a time to other vessels for subsequent processing in step 690. When one cell colony is being transferred, colonies in other compartments are submerged in the trypsin solution to avoid exposure to air and dry-out. In a conventional petri-dish design, the trypsin solution must be discarded before the transfer. While one cell colony is being transferred, other colonies in the petri-dish are exposed to air. Some of them may dry out and die eventually.

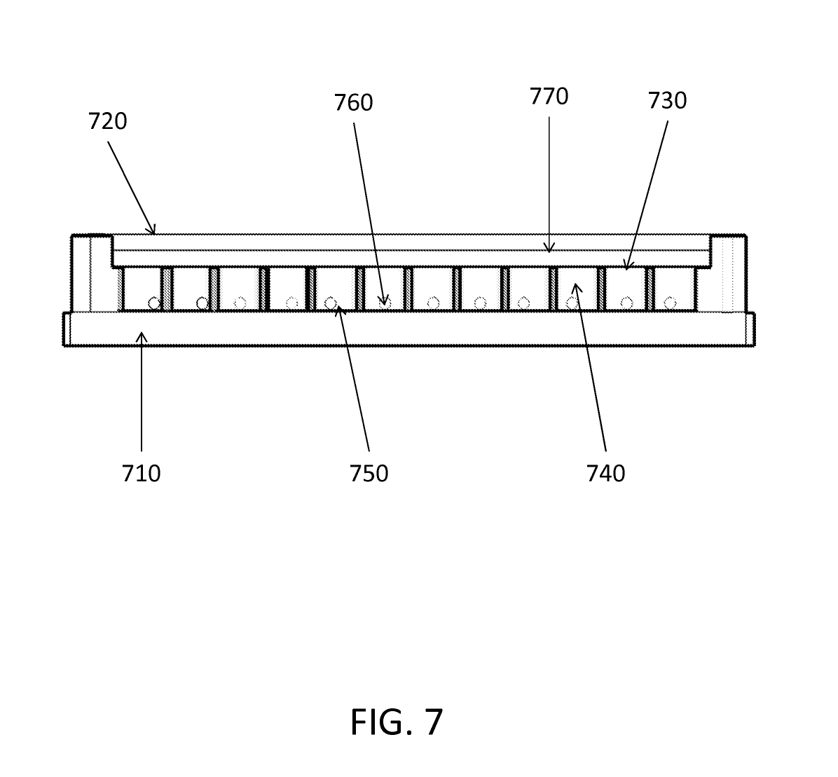

Referring to FIG. 7 now. FIG. 7 illustrates a plating position of the multi-compartment device 710 in the cell cloning process. The cell suspension solution level 770 is above the top openings 730 of the compartments 740. The extended sidewall 720 of the multi-compartment device in the present invention, which is at a height above the top openings 730 of the compartments 740, ensures the compartments 740 in the entire device 710 are overflown and filled with the cell suspension solution. The living cells 760 in the suspension settle to a bottom surface 750 of the compartments 740 and attach to the surface 750 to grow and split to form cell colonies.

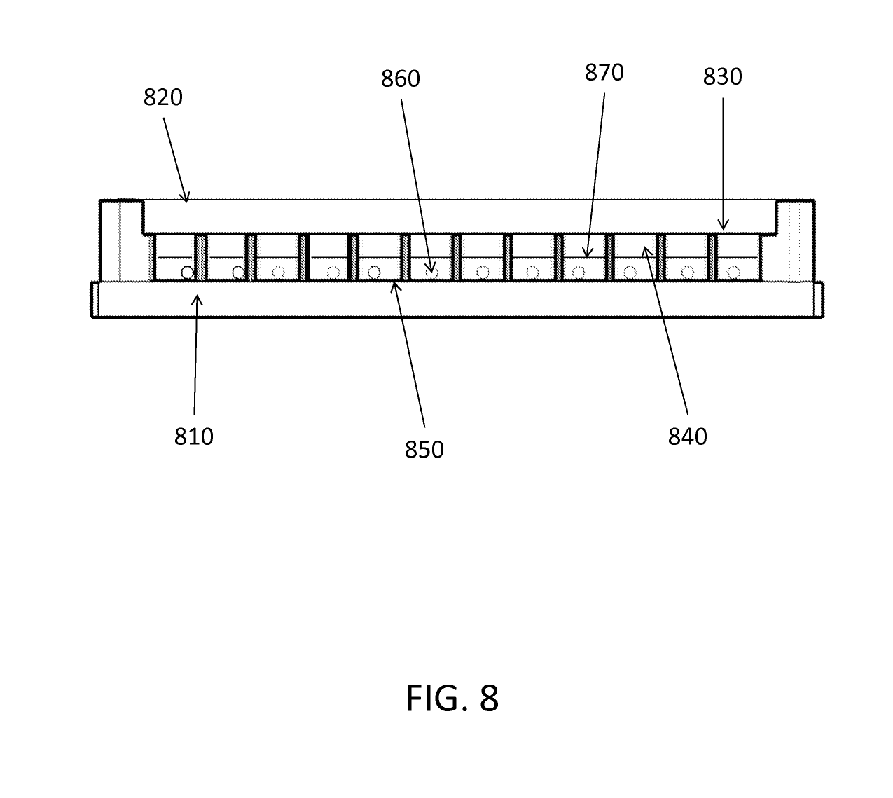

Referring to FIG. 8 now. In contrast, in the multi-compartment device position in the cell colony release and detachment process, as illustrated in FIG. 8, a trypsin solution is added to the multi-compartment device 810 to a level 870, which is below the top openings 830 of the compartments 840. The trypsin may be poured into the device without worries about spilling, thanks to the extended sidewall 820 of the multi-compartment device in the present invention. The trypsin cleaves the cell colonies 860 from a bottom surface 850 of the compartments 840. After releasing and detachment, the cell colonies are still confined in their individual compartments and do not float to adjacent compartments to cause contaminations therein. It is important that the cell colonies are still submerged under the trypsin at this stage to avoid exposure to air and dry-out. The cell colonies may be transferred individually to avoid contaminations. After the cell colonies in the entire multi-compartment device have been transferred, the device may be discarded and the cell cloning process is complete.

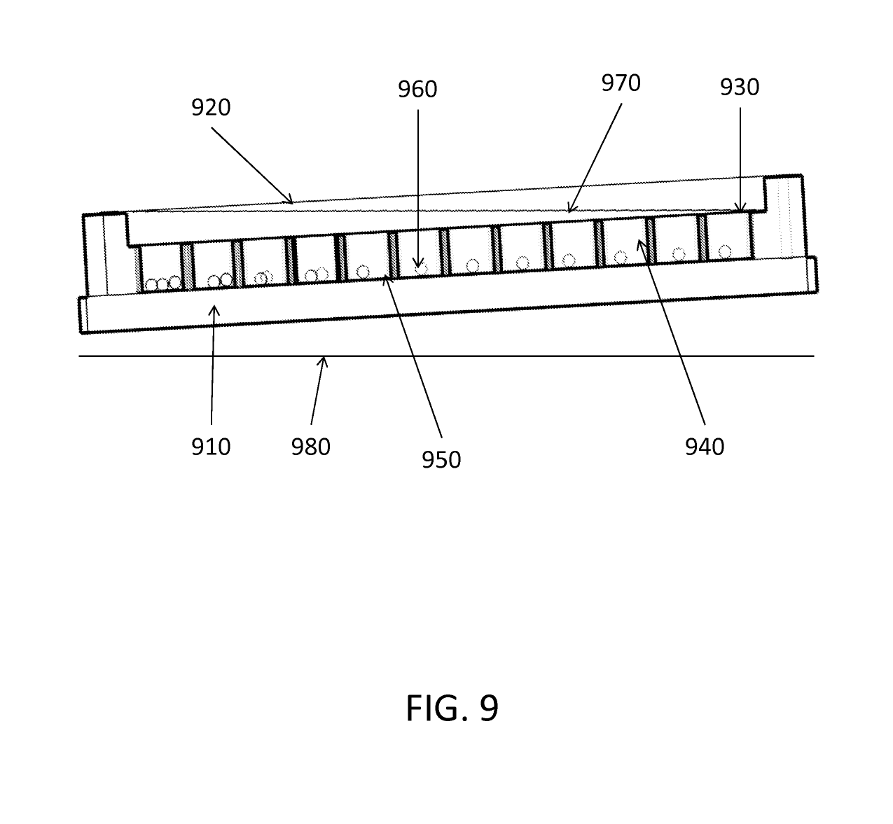

Referring to FIG. 9 now. The multi-compartment device in the present invention offers another unique advantage compared to conventional cell cloning devices. Illustrated in FIG. 9, during the plating process discussed heretofore, the multi-compartment device 910 may be tilted to a position such that one end of the device is taller than the other. The multi-compartment device 910 is at an angle to a flat surface 980. The cells 960 in the cell suspension solution flow to the lower end of the device 910 due to gravity effect. The extended sidewall 920 of the device 910 in the present invention maintains the suspension solution within the device 910 such that even though the suspension solution level 970 is taller at one end of the device 910 than the other, no spilling of the suspension solution would occur. This would not be possible with a conventional multi-well microplate. The cells 960 settle to a bottom surface 950 of the compartments 940 by gravity and attach onto the surface 950 to form cell colonies. Due to the tilting of the multi-compartment device 910, the cell suspension solution at the lower end of the device 910 has taller cell density than that of the solution at the taller end in the device 910. As a result, more cells 960 will occupy the compartments 940 at the lower end of the device 910 than at the taller end of the device 910. This is important when an optimal cell density of the suspension is unknown to ensure a taller possibility that at the taller end of the device 910 only one cell occupies each compartment to grow a single cell colony therein.

What is claimed is:

1. A method for cell cloning, comprising the steps of:
preparing a medium solution, said solution having a density lower than cells;
mixing said cells with said medium solution to form a suspension such that said cells are uniformly distributed in said suspension to a desired cell density;
filling a cell cloning device with said suspension, wherein a cell culture surface of said device is divided to form compartments;
overflowing said compartments with said suspension;
gravitating said cells to said culture surface;
culturing said cells to form cell colonies in said compartments;
discarding said medium solution, leaving a small amount of said medium solution in said compartments such that said cell colonies do not expose to air;
detaching said cell colonies; and
transferring said cell colonies to a container.

2. The method in claim 1, further comprising seeding a desired number of said cells to said culture surface of said compartments.

3. The method in claim 1, further comprising tilting said device such that said cells form a gradient in said suspension.

4. The method in claim 1, further comprising preparing said suspension to a cell density such that one survivable cell is settled in at least one of said compartments.

5. The method in claim 1, further comprising adding a selection reagent in said medium solution to annihilate cells nonresistant to said reagent and allowing cells resistant to said reagent to grow in said device.

6. A multi-compartment cell cloning device, comprising:
a body member having a top surface and a sidewall, whereby said sidewall is taller than said inner surface;
a plurality of compartments, formed inside said body member, a wall of said compartments extending upwardly such that a top opening of at least one of said compartments is formed on said top surface of said body member, said compartments adapted to receive at least one cell for cell cloning.

7. The device in claim 6, wherein said sidewall is at least 0.1 millimeter taller than said top surface of said body member.

8. A cell cloning device, comprising:
a body member having a top surface;
a plurality of compartments having a bottom surface, a wall of at least one of said compartments extending upwardly from said bottom surface such that a top opening of said compartments is formed on said top surface of said body member, said compartments adapted to receive at least one cell for cell cloning; and
a base member having an inner surface and a sidewall, said sidewall extending upwardly from said inner surface, whereby said body member engages in said base member and said sidewall is taller than said top surface of said body member.

9. The device in claim 8, further comprising said compartments in different sizes.

10. The device in claim 8, further comprising said compartments in different shapes.

11. The device in claim 8, further comprising multiple body members being assembled in said base member.

12. The device in claim 8, wherein said sidewall of said base member is at least 0.1 millimeter taller than said top surface of said body member when said body member and said base member are assembled.

13. A device for cell cloning, comprising:
a body member having a top surface and a bottom surface;
a plurality of compartments, a wall of at least one of said compartment extending upwardly such that a top opening of each of said compartments is formed on said top surface of said body member, a second opening of said compartments is formed on said bottom surface of said body member, said compartments adapted to receive at least one cell for cell cloning; and
a base member having an inner surface and a sidewall, said sidewall extending upwardly from said inner surface, whereby said body member engages in said base member and said sidewall is taller than said top surface of said body member.

14. The device in claim 13, further comprising said compartments in different sizes.

15. The device in claim 13, further comprising said compartments in different shapes.

16. The device in claim 13, further comprising multiple body members being assembled in said base member.

17. The device in claim 13, wherein said second opening of said compartments is attached to said inner surface of said base member when said body member and said base member are assembled.

18. The device in claim 13, wherein said sidewall of said base member is at least 0.1 mm taller than said top surface of said body member when said body member and said base member are assembled.

19. The device in claim 13, further comprising a seal means between said second openings and said inner surface to prevent cross-contamination.

20. The device in claim 13, further comprising at least one locking means for positioning said body member in place when said body member and said base member are assembled, to prevent movements of said body member and cross-contamination between said compartments.

21. The locking means in claim 20, wherein said locking means is a wedge formed on said sidewall of said base member, said wedge having a sloped first surface, said wedge having a second surface which is parallel to said inner surface of said base member, said second surface is above said body member such that said wedge presses against said top surface of said body member, when said body member and said base member are assembled.

22. The locking means in claim 20, further comprising said locking means being a recess formed on said sidewall of said base member and a protrusion formed on said body member, said protrusion inserting in said recess, when said body member and said base member are assembled.

23. The locking means in claim 20, further comprising said locking means being a protrusion formed on said sidewall of said base member and a recess formed on said body member, said protrusion inserting in said recess, when said body member and said base member are assembled.
